SEQUENCE LISTING (23 sequences)

SEQ ID NO: 1 - DNA, 16 bp, Artificial Sequence
  PNA probe
acggtgatattgctgt

SEQ ID NO: 2 - DNA, 19 bp, Artificial Sequence
  PNA probe
ggcgattcattaatttatg

SEQ ID NO: 3 - DNA, 16 bp, Artificial Sequence
  PNA probe
ttacaagttcaagcag

SEQ ID NO: 4 - DNA, 18 bp, Artificial Sequence
  PNA probe
gtcgtgatagtaagttcc

SEQ ID NO: 5 - DNA, 14 bp, Artificial Sequence
  PNA probe
gctctccttgcagg

SEQ ID NO: 6 - DNA, 14 bp, Artificial Sequence
  PNA probe
tgctatggggcgat

SEQ ID NO: 7 - DNA, 13 bp, Artificial Sequence
  PNA probe
tgacagcgcggtt

SEQ ID NO: 8 - DNA, 13 bp, Artificial Sequence
  PNA probe
gcgacatcgcctg

SEQ ID NO: 9 - DNA, 18 bp, Artificial Sequence
  PNA probe
cggtttatgacactatcg

SEQ ID NO: 10 - DNA, 19 bp, Artificial Sequence
  primer
cttccagatgtgcgtgatg

SEQ ID NO: 11 - DNA, 19 bp, Artificial Sequence
  primer
cgaagtgagaacggctgag

SEQ ID NO: 12 - DNA, 20 bp, Artificial Sequence
  primer
agtgtaatcgtggccagagc

SEQ ID NO: 13 - DNA, 18 bp, Artificial Sequence
  primer
caccgtcaccatccatag

SEQ ID NO: 14 - DNA, 21 bp, Artificial Sequence
  primer
cgtgatttgattacaatgttg

SEQ ID NO: 15 - DNA, 20 bp, Artificial Sequence
  primer
ccactacgggttacacttac

SEQ ID NO: 16 - DNA, 20 bp, Artificial Sequence
  primer
cggcaagaagaacaagaagg

SEQ ID NO: 17 - DNA, 20 bp, Artificial Sequence
  primer
atatgggcaccatcggtatc

SEQ ID NO: 18 - DNA, 18 bp, Artificial Sequence
  primer
ctgggcaatgactggaac

SEQ ID NO: 19 - DNA, 17 bp, Artificial Sequence
  primer
gccatgcgcacttcggt

SEQ ID NO: 20 - DNA, 20 bp, Artificial Sequence
  primer
cgtaaccgtaagaatcaggc

SEQ ID NO: 21 - DNA, 20 bp, Artificial Sequence
  primer
ctgtggtagcgcagcttatc

SEQ ID NO: 22 - DNA, 20 bp, Artificial Sequence
  primer
gtgtatgcgatgtctgagct

SEQ ID NO: 23 - DNA, 19 bp, Artificial Sequence
  primer
ggatagcgataggagaagg

(19) United States
(12) Patent Application Publication
(10) Pub. No.: US 2019/0177771 A1
(43) Pub. Date: Jun. 13, 2019

(54) PNA PROBE FOR DISCRIMINATION OF QUINOLONE ANTIBIOTIC RESISTANT BACTERIA AND METHOD FOR DISCRIMINATION OF ANTIBIOTIC RESISTANT BACTERIA USING THE SAME

(71) Applicant: REPUBLIC OF KOREA (National Institute of Fisheries Science), Busan (KR)

(72) Inventors: Myoung Sug Kim, Busan (KR); KyoungMi Won, Busan (KR); Jeong Wan Do, Busan (KR); Deok-Chan Lee, Busan (KR); Hyun-Ja Han, Busan (KR); Na Young Kim, Busan (KR); Miyoung Cho, Busan (KR); Sung-hee Jung, Busan (KR)

(21) Appl. No.: 16/198,904

(22) Filed: Nov. 23, 2018

(30) Foreign Application Priority Data
Dec. 8, 2017 (KR) 10-2017-0168362

(51) Int. Cl. C12Q 1/689; C12Q 1/6806; C12Q 1/686

(52) U.S. Cl. CPC C12Q 1/689; C12Q 1/6806; C12Q 2600/166; C12Q 2600/16; C12Q 2600/156; C12Q 1/686

(57) ABSTRACT

A PNA probe for discrimination or detection of quinolone antibiotic-resistant bacteria and a method for discrimination or detection of quinolone antibiotic-resistant bacteria are described. The method may be carried out by selecting and amplifying a quinolone antibiotic-resistance genetic marker of Vibrio harveyi, Streptococcus parauberis or Edward tarda, hybridizing to the amplification product a PNA that specifically recognizes the amplification product, obtaining a temperature-dependent melting curve while controlling the temperature of the hybridized product, and analyzing the melting curve to determine a melting temperature, thereby discriminating or detecting quinolone antibiotic-resistant bacteria. Accordingly, whether or not Vibrio harveyi, Streptococcus parauberis or Edward tarda is resistant to quinolone antibiotics can be determined in a simple, rapid and accurate manner, to assist efforts to reduce damage caused by vibriosis, streptococcosis or edwardsiellosis, which is an infectious aquatic organism disease caused by Vibrio harveyi, Streptococcus parauberis or Edward tarda.

### CROSS-REFERENCE TO RELATED APPLICATION

The priority under 35 USC § 119 of Korean Patent Application 10-2017-0168362 filed Dec. 8, 2017 is hereby claimed. The disclosure of Korean Patent Application 10-2017-0168362 is hereby incorporated herein by reference, in its entirety, for all purposes.

### TECHNICAL FIELD

The present invention relates to a PNA probe for discrimination or detection of quinolone antibiotic-resistant bacteria and a method for discrimination or detection of quinolone antibiotic-resistant bacteria using the same, and more particularly to a PNA probe for determining whether Vibrio harveyi, Streptococcus parauberis and Edward tarda are resistant to quinolone antibiotic, and a composition, a kit and a method for discrimination or detection of quinolone antibiotic-resistant bacteria using the same.

### BACKGROUND ART

The olive flounder aquaculture industry in South Korea has been undergoing rapid development, since the aquaculture production technology was established in the early 1980s and the land-based aquaculture method was developed. In 2011, the aquaculture production of olive flounder in South Korea was 40,805 M/T, which reached about 56% of the total aquaculture production of fish in Korea. Thus, olive flounder aquaculture has become a representative aquaculture industry in Korea (Statistics Korea, 2011).

Survival rate is the most important determinant of economic efficiency in the olive flounder aquaculture industry as well as other species. In the late 2000s, the culture environment of the olive flounder was deteriorated due to the lack of quality control, the poor quality of the water, the feed, the seedlings including bad seeds, and lack of farm management. In 2012, the total damage rate was 27.18% in the period of May to October nationwide in South Korea, of which 22.64% were caused by infectious diseases and 4.53% were caused by intentional or non-infectious causes. The causes of the damage were 35.9% of Scutica's disease, 19.6% of non-infectious diseases, 12.8% of streptococcosis, 9.2% of viral hemorrhagic sepsis, 6.9% of intentional damage, 6.7% of vibriosis, 2.9% of flavobacteriosis, 2.8% of white spot disease, 2.1% of edwardsiellosis.

The damage caused by bacterial infection is increasing, and the use of antibiotics to prevent this infection is also increasing. Antibiotics have been frequently used in animal husbandry and fisheries in order to prevent diseases, promote growth, or treat diseases. However, overuse of antibiotics has increased the incidence of antibiotic-resistant bacteria, and the antibiotic-resistant bacteria can be spread to humans through livestock and fishery products. For this reason, continuous monitoring of the antibiotic-resistant bacteria is required. Antibiotic-resistant bacteria are increasing worldwide with the threat of new infectious diseases, and rapid spread of antibiotic-resistant bacteria is emerging as a big problem in many countries of the world.

Methods for detecting antibiotic-resistant bacteria include a measuring MIC (minimal inhibitory concentration), a observing the phenotype of antibiotic-resistant bacteria using disc diffusion technique, and the like. However, in these detection methods, the time required for the detection tends to increase arithmetically as the amount of the sample increases, and thus when many samples are to be tested, a lot of time is taken to obtain the results. In addition, it is probable that subjective judgment is required, and errors between testing institutions may occur.

Accordingly, the present inventors have made extensive efforts to develop a method for discrimination or detection of the quinolone antibiotic resistance of Vibrio harveyi, Streptococcus parauberis or Edward tarda, which causes vibriosis, streptococcosis or edwardsiellosis, which is a major infectious disease in olive flounder. As a result, the present inventors have found that when information on the quinolone antibiotic resistance gene of Vibrio harveyi, Streptococcus parauberis or Edward tarda is identified and analysis is performed using a specific peptide nucleic acid and primer pair, whether or not the bacteria are resistant to antibiotics can be determined in a rapid and accurate manner, thereby determining the present invention.

The information disclosed in the Background Art section is only for the enhancement of understanding of the background of the present invention, and therefore may not contain information that forms a prior art that would already be known to a person of ordinary skill in the art.

### DISCLOSURE OF INVENTION

### Technical Problem

An object of the present invention is to provide a PNA probe for discrimination or detection of quinolone antibiotic-resistant bacteria.

Another object of the present invention is to provide a composition and a kit for discrimination or detection of quinolone antibiotic-resistant bacteria, which comprises the PNA probe.

Still another object of the present invention is to provide a method for discrimination or detection of quinolone antibiotic-resistant bacteria using the PNA probe.

### Technical Solution

To achieve the above object, the present invention provides a PNA probe for discrimination or detection of quinolone antibiotic-resistant bacteria which is represented by any one nucleotide sequence selected from the group consisting of SEQ ID NOS: 1 to 9.

The present invention also provides a composition and a kit for discrimination or detection of quinolone antibiotic-resistant bacteria, comprising: a primer pair capable of amplifying one or more genes selected from the group consisting of gyrA, gyrB, and parC, which contain a single nucleotide polymorphism (SNP); and the PNA probe.

The present invention also provides a method for discrimination or detection of quinolone antibiotic-resistant bacteria, comprising the steps of: (a) extracting a target nucleic acid from a sample; (b) amplifying a quinolone antibiotic-resistant genetic marker nucleotide sequence contained in the target nucleic acid, as a template, by use of a primer pair capable of amplifying one or more genes selected from the group consisting of gyrA, gyrB, and parC, which contain an SNP, and hybridizing the PNA probe to the amplified genetic marker nucleotide sequence; (c) obtaining a temperature-dependent melting curve while increasing the temperature of the PNA probe-hybridized product resulting from step (b); and (d) analyzing the melting curve obtained in step (c) to determine a melting temperature, thereby detecting whether or not the bacteria are resistant to the quinolone antibiotic.

### Advantageous Effects

According to the present invention, whether or not Vibrio harveyi, Streptococcus parauberis or Edward tarda is resistant to quinolone antibiotics can be determined in a simple, rapid and accurate manner through selection of a quinolone antibiotic-resistance genetic marker of Vibrio harveyi, Streptococcus parauberis or Edward tarda, which is a major causative bacteria of infectious aquatic organism diseases, amplification of a nucleotide sequence of the selected genetic marker using a peptide nucleic acid and a primer specific for the genetic marker, and analysis of a temperature-dependent melting curve. Thus, the present invention is useful for reducing damage caused by vibriosis, streptococcosis or edwardsiellosis, which is caused by Vibrio harveyi, Streptococcus parauberis or Edward tarda.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic view showing the structural characteristic of a PNA probe.

FIG. 2 is a schematic view showing a step of obtaining a melting curve by hybridization of a peptide nucleic acid in a method for discrimination or detection of antibiotic-resistant bacteria.

FIGS. 3 to 9 show nucleotide sequence views illustrating the nucleotide sequences of a portion and SNP of each of quinolone antibiotic resistance genes and peptide nucleic acids derived therefrom.

FIGS. 10 to 12 are gene position views illustrating major nucleotide mutation sites included in peptide nucleic acids on quinolone antibiotic-resistance gene amplification products.

FIG. 13 shows real-time PCR reaction conditions for discrimination and detection of antibiotic-resistant bacteria.

FIG. 14 shows amplification curve graphs of S. parauberis, E. tarda, and V. harveyi, obtained using peptide nucleic acid probes and primer pairs for discrimination or detection of quinolone antibiotic-resistant bacteria.

FIG. 15 shows amplification curve and melting curve graphs of S. parauberis, E. tarda, and V. harveyi, obtained using peptide nucleic acid probes and primer pairs for discrimination or detection of quinolone antibiotic-resistant bacteria.

FIGS. 16 to 18 show temperature-dependent melting curve graphs of S. parauberis, E. tarda, and V. harveyi, obtained using peptide nucleic acid probes and primer pairs for discrimination and detection of quinolone antibiotic-resistant bacteria.

FIG. 19 is a table listing scores at different melting temperatures (Tₘ), which may be used to determine whether or not bacteria are resistant to antibiotics.

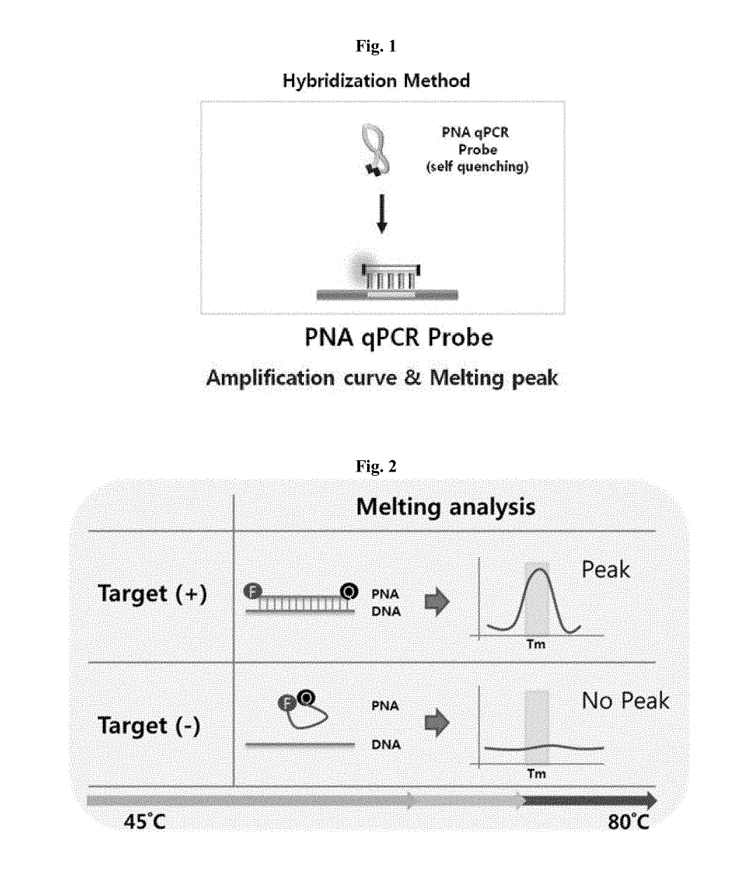

### BEST MODE FOR CARRYING OUT THE INVENTION

Unless defined otherwise, all technical and scientific terms used herein have the same meaning as commonly understood by one of ordinary skill in the art to which the invention pertains. Generally, the nomenclature used herein and the experiment methods, which will be described below, are those well known and commonly employed in the art.

In an example of the present invention, a genetic marker having quinolone antibiotic resistance was selected using a Vibrio harveyi, Streptococcus parauberis or Edward tarda sample, and quinolone antibiotic-resistant bacteria could be discriminated using a primer pair and PNA probe corresponding to the genetic marker. More specifically, from a DNA nucleotide sequence encoding a quinolone antibiotic resistance, a genetic marker containing a single nucleotide polymorphism (SNP) was selected as a target, and quinolone antibiotic-bacteria could be detected and discriminated using a primer pair and a PNA probe for discrimination of quinolone antibiotic-bacteria, which correspond to the genetic marker.

Therefore, in one aspect, the present invention is directed to a probe for discrimination or detection of quinolone antibiotic-resistant bacteria, which is represented by any one nucleotide sequence selected from the group consisting of SEQ IDS NOs: 1 to 9.

In the present invention, the PNA probe may have a reporter and a fluorescence quencher attached to both ends. The fluorescence quencher can quench the fluorescence of the reporter. The reporter may be one or more selected from the group consisting of FAM (6-carboxyfluorescein), Texas red, HEX (2′,4′,5′,7′,-tetrachloro-6-carboxy-4,7-dichlorofluorescein), JOE, Cy3 and Cy5. The quencher may be one or more selected from the group consisting of TAMRA (6-carboxytetramethyl-rhodamine), BHQ1, BHQ2 and Dabcyl, but is not limited thereto and preferably Dabcyl (FAM-labeled) can be used as the quencher.

Peptide nucleic acid (PNA) is a DNA analogue having nucleotides connected by peptide bonds, but not phosphate bonds, and was first synthesized by Nielsen et al. in 1991. PNA is artificially synthesized by a chemical method, but not found in natural systems.

Peptide nucleic acid is one of substances that recognize genes, like LNA (locked nucleic acid) or MNA (morpholino nucleic acid). It is artificially synthesized and has a backbone consisting of polyamide. PNA has advantages in that it has very high affinity and selectivity, and has a high stability for nuclease so that it is not cleaved by an existing restriction enzyme. In addition, PNA advantageously is thermally and chemically highly stable so that it is easily stored and is not readily degraded.

PNA forms a duplex by its hybridization to a natural nucleic acid having a nucleotide sequence complementary thereto. When their lengths are equal, a PNA/DNA duplex is more stable than a DNA/DNA duplex, and a PNA/RNA duplex is more stable than a DNA/RNA duplex. Furthermore, since PNA has a single base mismatch that makes the duplex unstable, the ability of PNA to detect SNP (single nucleotide polymorphism) is better than that of natural nucleic acid. Furthermore, PNA-DNA binding affinity is much higher than DNA-DNA binding affinity, and thus there is a difference in melting point of about 10 to 15° C. even in the presence of one nucleotide mismatch. Using this difference in binding affinity, changes in SNP and In/Del nucleotides can be detected.

Although the length of the PNA nucleotide sequence according to the present invention is not particularly limited, it may be constructed to have a length of 12 to 18 mer so as to contain a specific nucleotide sequence (e.g., the SNP) depending on viral species. In the present invention, a PNA probe may be designed to have a desired Tₘ value by adjusting the length of the PNA probe, and even in the case of PNA probes having the same length, the Tₘ value may be adjusted by changing the nucleotide sequence. Furthermore, since a PNA probe has a binding affinity higher than a DNA probe, it has a higher Tₘ value. Thus, the PNA probe can be designed to have a length shorter than a DNA probe, so that it can detect even adjacent nucleotide variation or SNP. In a conventional high-resolution melt (HRM) method, a difference in Tₘ value is very small (about 0.5° C.). For this reason, when two or more nucleotide variations appear, they cannot match with nucleotide sequence variations. However, the PNA probe according to the present invention shows a distinct difference in Tₘ values between nucleotide positions, and thus can be analyzed.

In the present invention, the quinolone antibiotic-resistant bacteria may be Vibrio harveyi, Streptococcus parauberis, or Edwardsiella tarda.

In one example of the present invention, the correlation between the MIC values of isolated V. harveyi strains for quinolone antibiotic and a point mutation of the gyrA gene was examined. The 248^{th} nucleotide in the gyrA gene of V. harveyi FP4541 having resistance to oxolinic acid and Flumequin changed from G to T, and the 83^{rd} amino acid of the gyrA protein changed from serine to isoleucine. The quinolone resistance-determining region (QRDR) of V. harveyi has not been reported. However, according to this example, it is believed that because the QRDR of V. vulnificus having resistance to quinolone antibiotic is the same as that of Photobacterium damselae subsp. piscicida, the 248^{th} nucleotide of the gyrA gene is the QRDR of V. harveyi (Table 4).

In one example of the present invention, it was shown that the 242^{nd} nucleotide of S. parauberis changed from C to T, and thus the 81^{st} amino acid changed from serine to leucine. In addition, no point mutation was observed in a region known as the quinolone resistance-determining region (QRDR) in the parC and parE genes (Table 2).

In one example of the present invention, for isolated E. tarda strains, the 249^{th} nucleotide in the gyrA gene changed from C to A, and the 83^{rd} amino acid of the gyrA protein changed from serine to arginine. The 248^{th} nucleotide of the FP3139 stain having the highest MIC value changed from G to T, and thus the 83^{rd} amino acid changed from serine to isoleucine. In addition, no point mutation was observed in a region known as the QRDR in the gyrB and parC genes (Table 3).

In another aspect, the present invention is directed to a composition for discrimination or detection of quinolone antibiotic-resistant bacteria, the composition comprising: a primer pair capable of amplifying one or more genes selected from the group consisting of gyrA, gyrB, and parC, which contain a single nucleotide polymorphism (SNP); and the PNA probe.

In the present invention, the primer pair may be a primer pair represented by any one nucleotide sequence pair selected from the group consisting of a nucleotide sequence pair of SEQ ID NOs: 10 and 11, a nucleotide sequence pair of SEQ ID NOs: 12 and 13, a nucleotide sequence pair of SEQ ID NOs: 14 and 15, a nucleotide sequence pair of SEQ ID NOs: 16 and 17, a nucleotide sequence pair of SEQ ID NOs: 18 and 19, a nucleotide sequence pair of SEQ ID NOs: 20 and 21, and a nucleotide sequence pair of SEQ ID NOs: 22 and 23.

In still another aspect, the present invention is directed to a kit for discrimination or detection of quinolone antibiotic-resistant bacteria, the kit comprising: a primer pair capable of amplifying one or more genes selected from the group consisting of gyrA, gyrB, and parC, which contain a single nucleotide polymorphism (SNP); and the PNA probe.

In the present invention, the primer pair may be primer pair represented by any one pair of nucleotide sequences selected from the group consisting of a pair of nucleotide sequences of SEQ ID NOS: 10 and 11, a pair of nucleotide sequences of SEQ ID NOS: 12 and 13, a pair of nucleotide sequences of SEQ ID NOS: 14 and 15, a pair of nucleotide sequences of SEQ ID NOS: 16 and 17, a pair of nucleotide sequences of SEQ ID NOS: 18 and 19, a pair of nucleotide sequences of SEQ ID NOS: 20 and 21, and a pair of nucleotide sequences of SEQ ID NOS: 22 and 23.

The kit of the present invention may optionally include reagents required for performing a target nucleic acid amplification reaction (e.g., PCR reaction), such as buffer, DNA polymerase cofactor, and deoxyribonucleotide-5-triphosphate. In addition, the kit of the present invention may also comprise various polynucleotide molecules, a reverse transcriptase, various buffers and reagents, and an antibody that inhibits the activities of a DNA polymerase. In addition, in the kit, the optimal amount of the reagent used in a specific reaction can be easily determined by those skilled in the art who have acquired the disclosure set forth herein. Typically, the kit of the invention may be manufactured as a separate package or compartment comprising the above mentioned ingredients.

When the kit is used, a single nucleotide mutation and a mutation caused by nucleotide deletion or insertion in a target nucleic acid can be effectively detected by analysis of a melting curve obtained using the PNA probe, so that whether or not the bacteria are resistant to quinolone antibiotic can be determined.

In yet another aspect, the present invention is directed to a method for discrimination or detection of quinolone antibiotic-resistant bacteria, comprising the steps of: (a) extracting a target nucleic acid from a sample; (b) amplifying a quinolone antibiotic-resistant genetic marker nucleotide sequence contained in the target nucleic acid, as a template, by use of a primer pair capable of amplifying one or more genes selected from the group consisting of gyrA, gyrB, and parC, which contain an SNP, and hybridizing the PNA probe to the amplified genetic marker nucleotide sequence; (c) obtaining a temperature-dependent melting curve while increasing the temperature of the PNA probe-hybridized product resulting from step (b); and (d) analyzing the melting curve obtained in step (d) to determine a melting temperature, thereby detecting whether or not the bacteria are resistant to the quinolone antibiotic.

In the present invention, the primer pair may be primer pair represented by any one pair of nucleotide sequences selected from the group consisting of a pair of nucleotide sequences of SEQ ID NOS: 10 and 11, a pair of nucleotide sequences of SEQ ID NOS: 12 and 13, a pair of nucleotide sequences of SEQ ID NOS: 14 and 15, a pair of nucleotide sequences of SEQ ID NOS: 16 and 17, a pair of nucleotide sequences of SEQ ID NOS: 18 and 19, a pair of nucleotide sequences of SEQ ID NOS: 20 and 21, and a pair of nucleotide sequences of SEQ ID NOS: 22 and 23.

As used herein, the term “sample” is meant to include various samples. Preferably, a biosample is analyzed using the method of the present invention. More preferably, the sample may be either a sample that is mixed with Vibrio harveyi, Streptococcus parauberis, or Edwardsiella tarda, or a sample from an individual (for example, fish or the like) infected with the bacteria. Biosamples originated from plants, animals, humans, fungi, bacteria and virus can be analyzed. When a mammal- or human-originated sample is analyzed, it may be derived from specific tissues or organs. Representative examples of tissues include connective tissue, muscle, or nerve tissue. Representative examples of organs include eyes, brain, lung, liver, spleen, bone marrow, thymus, heart, lymph, blood, bone, cartilage, pancreas, kidney, gallbladder, stomach, small intestine, testis, ovary, uterus, rectum, nervous system, and gland and internal blood vessels. A biosample to be analyzed includes any cell, tissue or fluid that is derived from a biological origin, or any other medium that can be well analyzed by the present invention. The biosample also includes a sample obtained from foods produced for consumption of humans and/or animals. In addition, the to-be-analyzed biosample includes a body fluid sample, which includes, but not limited to, blood, serum, plasma, lymph, breast milk, urine, feces, ocular fluid, saliva, semen, brain extracts (e.g., grinded brain), spinal fluid, appendix, spleen, and tonsil tissue extracts, but not limited thereto.

As used herein, the term “target nucleic acid” means a nucleic acid sequence (containing nucleotide variation or SNP) to be detected. The target nucleic acid comprises a specific region of the nucleic acid sequence of a “target gene” encoding a protein having physiological and biochemical functions, and is annealed or hybridized to the primer or the probe under annealing, hybridization, or amplification conditions.

In the present invention, the ‘amplification’ may be performed by a real-time polymer chain reaction (PCR) method, but is not limited thereto.

In the real-time PCR method according to the present invention, a fluorescent substance is interchelated into a double-stranded DNA duplex during PCR, and the temperature is increased together with amplification to melt the DNA double strands to thereby reduce the amount of fluorescent substance present between the DNA double strands. The resulting melting curve pattern, particularly the temperature (Tₘ) at which the DNA is melted (denatured), may be analyzed, thereby determining or detecting antibiotic-resistant bacteria based on the presence or absence of the specific nucleotide sequence.

A specific nucleotide sequence (e.g., SNP) analysis using the PNA probe can be sufficiently achieved using a forward/reverse primer pair for PCR and a probe comprising nucleotide(s) that recognize(s) the specific nucleotide sequence, and a primer of producing a single-strand genetic marker sequence fragment using a genetic marker nucleotide sequence amplified by the primer set as a template. The PCR may be performed using a conventional method, and after completion of the PCR, a melting process is required. Whenever the melting temperature increases by 0.5 to 1° C., the intensity of fluorescence is measured to obtain the Tₘ value. In particular, general real-time PCR systems are widely known and have an advantage in that purchase of an additional program such as a HRM (high-resolution melting) program or a minute temperature change is not required.

As used herein, the term “hybridization” means that complementary single-stranded nucleic acids form a double-stranded nucleic acid. Hybridization can occur when the complementarity between two nucleic acid strands is perfect (perfect match) or when some mismatched residues exist. The degree of complementarity necessary for hybridization may vary depending on hybridization conditions, particularly may be controlled by temperature.

In the present invention, the PNA probe may have a reporter and a fluorescence quencher attached to both ends. The fluorescence quencher can quench the fluorescence of the reporter. The reporter may be one or more selected from the group consisting of FAM (6-carboxyfluorescein), Texas red, HEX (2′,4′,5′,7′,-tetrachloro-6-carboxy-4,7-dichlorofluorescein), JOE, Cy3, and Cy5. The quencher may be one or more selected from the group consisting of TAMRA (6-carboxytetramethyl-rhodamine), BHQ1, BHQ2 and Dabcyl, but is not limited thereto and preferably Dabcyl (FAM-labeled) can be used as the quencher.

The PNA probe comprising the reporter and the quencher according to the present invention generates a fluorescent signal after its hybridization to the target nucleic acid. As the temperature increases, the PNA probe is rapidly melted with the target nucleic acid at its suitable melting temperature, and thus the fluorescent signal is quenched. Through analysis of a high-resolution melting curve obtained from the fluorescent signal according to temperature changes, the presence or absence of a nucleotide modification (including SNP) of the target nucleic acid may be detected. If the PNA probe perfectly matches with the nucleotide sequence of the target nucleic acid, it then shows an expected melting temperature (Tₘ) value, but if the PNA probe mismatches with a target nucleic acid in which a nucleotide mutation is present, it shows a melting temperature (Tₘ) value lower than an expected value.

As used herein, the term “nucleotide variation” refers to a change in a nucleotide sequence of a target nucleic acid (e.g., a substitution, deletion or insertion of one or more nucleotides, as well as a single nucleotide polymorphism (SNP)) relative to a reference sequence. The PNA probe of the present invention can analyze a change in a nucleotide sequence of a target nucleic acid such as, SNP of the target nucleic acid or a substitution, deletion or insertion of nucleotides of the target nucleic acid through the melting curve analysis.

The Tₘ value also changes depending on the difference between the nucleotide sequence of the PNA probe and the nucleotide sequence of a DNA complementary thereto, and thus the development of applications based on this change is easily achieved. The PNA probe is analyzed using a hybridization method different from a hydrolysis method used for a TaqMan probe, and probes having functions similar to that of the PNA probe include molecular beacon probes and scorpion probes.

In the present invention, the ‘melting curve analysis’ is a method of analyzing a double-strand nucleic acid formed of the target nucleic acid DNA or RNA and the probe. This method is called melting curve analysis, because it is performed by, for example, Tₘ analysis or the analysis of the melting curve of the double-strand nucleic acid. Using a probe complementary to a specific nucleotide sequence (including nucleotide variation or SNP) of a target to be detected, a hybrid (double-strand DNA) of a target single-strand DNA of a sample to be detected and the probe is formed. Subsequently, the formed hybrid is heated, and the dissociation (melting) of the hybrid, which results from an increase in the temperature, is detected based on a change in a signal such as absorbance. Based on the results of the detection, the Tₘ value is determined, so that the presence or absence of the specific nucleotide sequence can be determined. The Tₘ value increases as the homology of the formed hybrid increases, and the Tₘ value decreases as the homology decreases. For this reason, the Tₘ value of a hybrid formed of a specific nucleotide sequence of a target to be detected and a probe complementary thereto is previously determined (a reference value for evaluation), and the Tₘ value of a hybrid formed of the target single-strand DNA of a sample to be detected and the probe is measured (a measured value). If the measured value is approximately equal to the reference value, it can be determined that the probe matches, that is, a specific nucleotide sequence is present in the target DNA. If the measured value is lower than the reference value, it can be determined that the probe mismatches, that is, a targeting nucleotide sequence is absent or a mutation is present in the target DNA.

In the present invention, the melting curve analysis may be performed by a fluorescence melting curve analysis (FMCA) method, but is not limited thereto.

The fluorescent melting curve analysis of the present invention is a method that analyzes a melting curve using a fluorescent material, and more specifically, may analyze the melting curve by using a probe containing a fluorescent material. The fluorescent material may be either a reporter or a quencher, and may preferably be an intercalating fluorescent material.

As a technical method for optimizing the results of the present invention, a liquid type U-TOP method (Seasun Biomaterials, Korea) may be used. This method is a liquid type array method which uses a PNA probe having attached thereto a reporter and a quencher, which can effectively detect a single nucleotide substitution, deletion or insertion of a target nucleic acid. This method does not require a washing process following a hybridization process, and also does not require a process of immobilizing the PNA probe onto a plate.

### EXAMPLES

Hereinafter, the present invention will be described in further detail with reference to examples. It will be obvious to a person having ordinary skill in the art that these examples are illustrative purposes only and are not to be construed to limit the scope of the present invention.

### Example 1: Selection of Antibiotic-Resistant Bacteria by Measurement of Minimal Inhibitory Concentration (MIC), and Analysis of Quinolone Antibiotic-Resistance Gene

Using S. parauberis, E. tarda, and V. harveyi samples stored in the National Institute of Fisheries Science, MIC values were measured. Antibiotics used for each strain are shown in Table 1 below.

TABLE 1
Kind of antibiotics used for discrimination or detection
of quinolone antibiotic-resistant bacteria

| Bacteria species | Antibiotics |
|---|---|
| S. parauberis | Cipfloxacin |
| E. tarda | Oxolinic acid, Flumequine, Cipfloxacin |
| V. harveyi | Oxolinic acid, Flumequine |

It was shown that the 242^{nd} nucleotide of S. parauberis changed from C to T, and thus the 81^{st} amino acid changed from serine to leucine. In addition, no point mutation was observed in a region known as the quinolone resistance-determining region (QRDR) in the parC and parE genes (Table 2).

TABLE 2
Comparison of MIC for ciprofloxacin and point mutations
in S. parauberis gyrA, parC and parE genes

| | | gyrA | | parC | | parE | |
|---|---|---|---|---|---|---|---|
| | MIC (μg/ml) | | Amino | | Amino | | Amino |
| Strain | Cipfloxacin | Nucleotide | acid | Nucleotide | acid | Nucleotide | acid |
| FP5227 | 0.06 | TCA | Ser⁸¹ | GAA | Glu⁷⁹ | CGT | Arg⁴⁴⁹ |
| FP3059 | 0.13 | TCA | Ser⁸¹ | GAA | Glu⁷⁹ | CGT | Arg⁴⁴⁹ |
| FP4031 | 0.25 | TCA | Ser⁸¹ | GAA | Glu⁷⁹ | CGT | Arg⁴⁴⁹ |
| FP4029 | 0.5 | TCA | Ser⁸¹ | GAA | Glu⁷⁹ | CGT | Arg⁴⁴⁹ |
| FP3227 | 1 | TCA | Ser⁸¹ | GAA | Glu⁷⁹ | CGT | Arg⁴⁴⁹ |
| FPa4533 | 2 | TTA | Leu⁸¹ | GAA | Glu⁷⁹ | CGT | Arg⁴⁴⁹ |

It was shown that the 249^{th} nucleotide in the gyrA gene of isolated E. tarda strains changed from C to A, and thus the 83^{rd} amino acid of the gyrA protein changed from serine to arginine. In addition, it was confirmed that the 248th nucleotide in the FP3139 strain having the highest MIC value changed from G to T, and thus the 83^{rd} amino acid changed to isoleucine. Furthermore, no point mutation was observed in a region known as the QRDR in the gyrB and parC genes (Table 3).

TABLE 3
Comparison of MIC for quinolone antibiotics and point
mutations in E. tarda gyrA, gyrB, and parC genes

| | MIC (μg/ml) | | | gyrA | | | | gyrB | | parC | |
|---|---|---|---|---|---|---|---|---|---|---|---|
| | Oxolinic | | | | Amino | | Amino | | Amino | | Amino |
| Strain | acid | Flumequine | Cipfloxacin | Nucleotide | acid | Nucleotide | acid | Nucleotide | acid | Nucleotide | acid |
| FP2240 | <0.25 | 1 | <0.06 | GAC | Asp⁸⁷ | AGC | Ser⁸³ | TCG | Ser⁴⁶⁴ | AGC | Ser⁸⁴ |
| FP2074 | 4 | 8 | 0.13 | GAC | Asp⁸⁷ | AGA | Arg⁸³ | TCG | Ser⁴⁶⁴ | AGC | Ser⁸⁴ |
| FP3071 | 8 | 16 | 0.25 | GAC | Asp⁸⁷ | AGA | Arg⁸³ | TCG | Ser⁴⁶⁴ | AGC | Ser⁸⁴ |
| FP2243 | 16 | 32 | 0.5 | GAC | Asp⁸⁷ | AGA | Arg⁸³ | TCG | Ser⁴⁶⁴ | AGC | Ser⁸⁴ |
| FP2130 | 64 | 64 | 1 | GAC | Asp⁸⁷ | AGA | Arg⁸³ | TCG | Ser⁴⁶⁴ | AGC | Ser⁸⁴ |
| FPa4445 | 8 | 32 | 2 | GAC | Asp⁸⁷ | AGA | Arg⁸³ | TCG | Ser⁴⁶⁴ | AGC | Ser⁸⁴ |
| FP3139 | 256 | ≥512 | 4 | GAC | Asp⁸⁷ | ATC | Ile⁸³ | TCG | Ser⁴⁶⁴ | AGC | Ser⁸⁴ |

The correlation between the MIC values of isolated V. harveyi strains and a point mutation of the gyrA gene was examined. It was shown that the 248^{th} nucleotide in the gyrA gene of V. harveyi FP4541 having resistance to oxolinic acid and Flumequin changed from G to T, and the 83^{rd} amino acid of the gyrA protein changed from serine to isoleucine. The quinolone resistance-determining region (QRDR) of V. harveyi has not been reported. However, according to this example, it is believed that because the QRDR of V. vulnificus having resistance to quinolone antibiotic is the same as that of Photobacterium damselae subsp. piscicida, the 248^{th} nucleotide of the gyrA gene is the QRDR of V. harveyi (Table 4).

TABLE 4
Comparison of MIC for quinolone antibiotics and point
mutation in V. harveyi gyrA gene

| | MIC (μg/ml) | | gyrA | |
|---|---|---|---|---|
| Strain | Oxolinic acid | Flumequine | Nucleotide | Amino acid |
| FP3023 | 1 | 1 | AGT | Ser⁸³ |
| FP3031 | 1 | 2 | AGT | Ser⁸³ |
| FP3457 | 2 | 2 | AGT | Ser⁸³ |
| FP7021 | 1 | 1 | AGT | Ser⁸³ |
| FP8045 | 1 | 2 | AGT | Ser⁸³ |
| FP4541 | 8 | 16 | ATT | Ile⁸³ |

### Example 2: Construction of PNA Probes and Primers for Discrimination or Detection of Quinolone Antibiotic-Resistant Bacteria

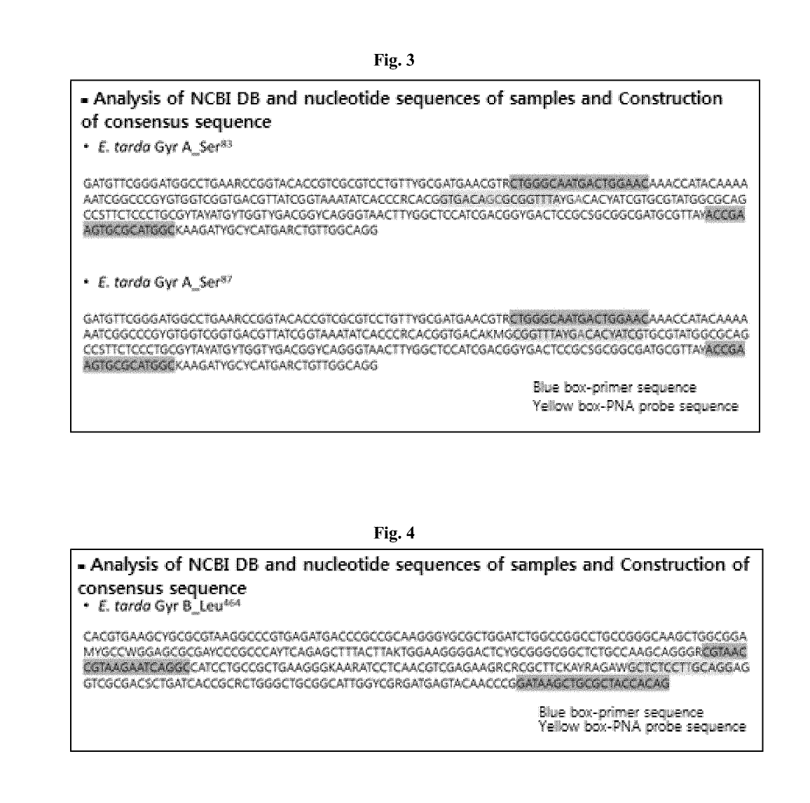

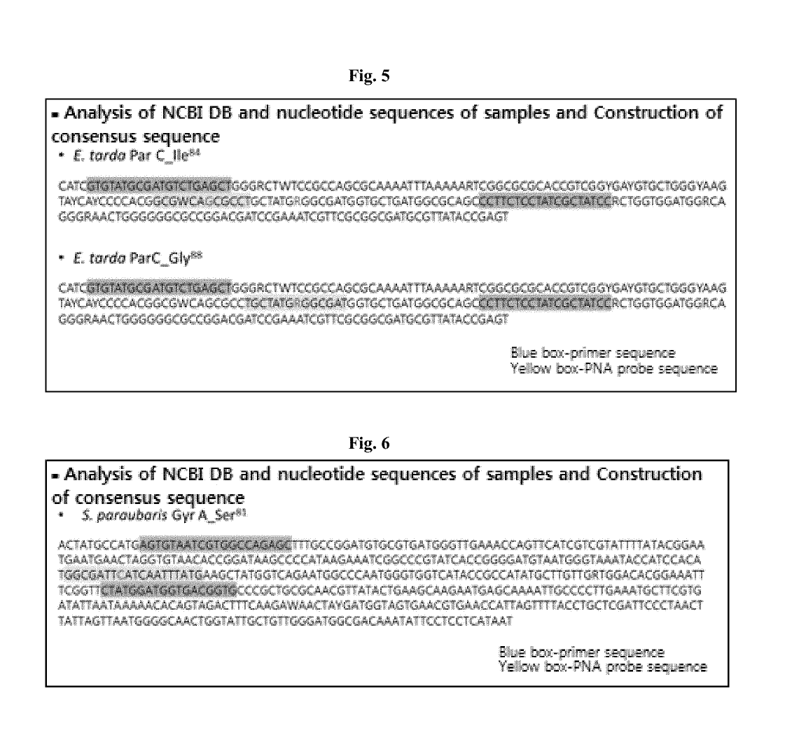

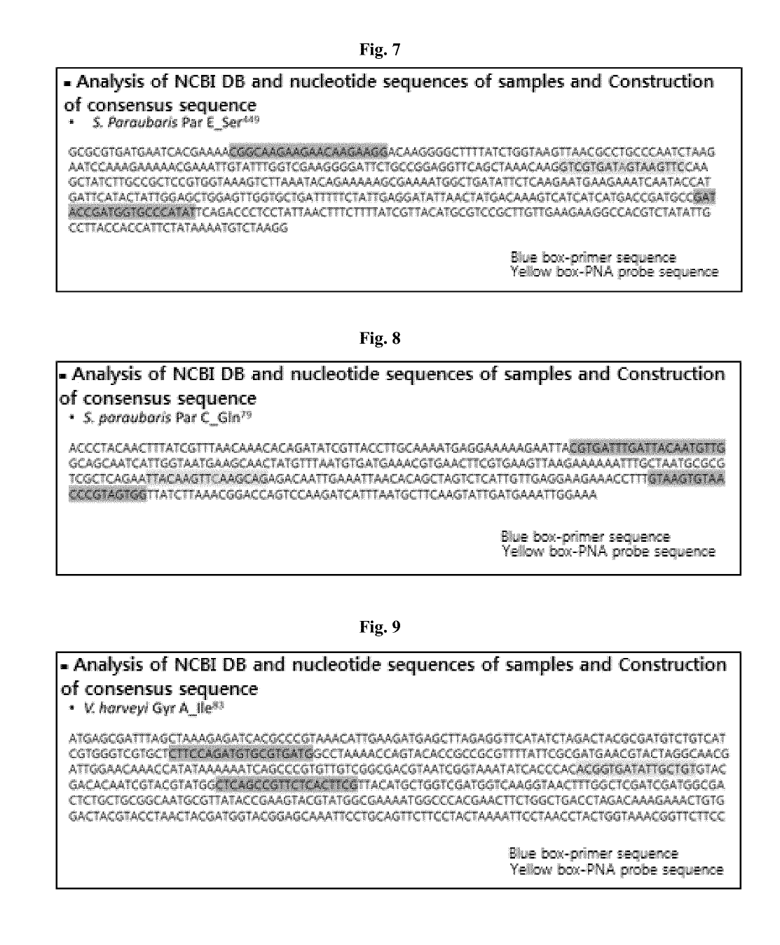

Based on the results of Example 1, a total of nine quinolone antibiotic-resistance genes were selected (FIGS. 3 to 9). FIGS. 3 to 9 are gene position views showing the nucleotide sequences of genes. In FIGS. 3 to 9, the PNA probe nucleotide sequence is shown in yellow shade, and the primer nucleotide sequence is indicated by blue bold letters.

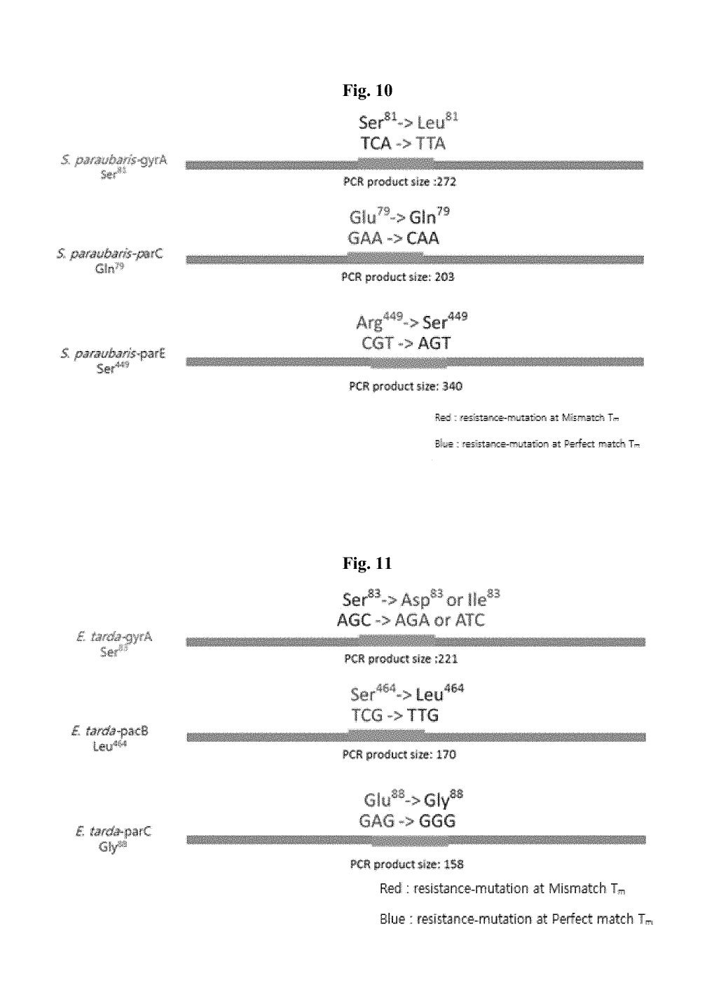

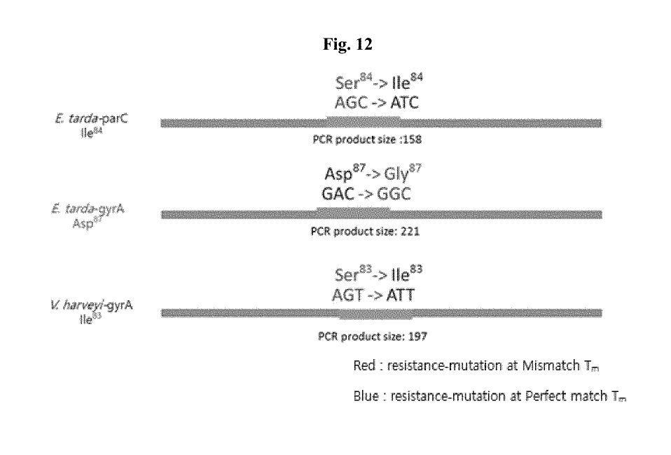

FIGS. 10 to 12 are gene position views showing gene mutation sites having quinolone antibiotic resistance, which include PNA probes on bacterial genes according to the present invention. Each nucleotide number is indicated based on the PCR product size of a gene which can be expressed as a protein.

Based on the analysis of collected date, quinolone antibiotic resistance genes were selected. To analyze a total of nine genes, PNA probes were designed (Table 5), and primer pairs for amplifying these genes were also constructed (Table 6).

TABLE 5
Information on nucleotide sequences of quinolone PNA
probes

| SEQ ID | | | |
|---|---|---|---|
| NOS: | Probe name | PNA probe Sequence (5′->3′) | Target gene |
| 1 | V. harveyi | Dabcyl-ACGGTGATATTGCTGT-O-K(Cy5) | V. harveyi |
| | GyrA_Ile⁸³ | | GyrA_Ile⁸³ |
| 2 | S. parauberis | Dabcyl-GGCGATTCATTAATTTATG-O- | S. parauberis |
| | GyrA_Ser⁸¹ | K(FAM) | GyrA_Ser⁸¹ |
| 3 | S. parauberis | Dabcyl-TTACAAGTTCAAGCAG-O- | S. parauberis |
| | ParC_Gln⁷⁹ | K(HEX) | ParC_Gln⁷⁹ |
| 4 | S. Parauberis | Dabcyl-GTCGTGATAGTAAGTTCC-O- | S. Parauberis |
| | ParE_Ser⁴⁴⁹ | K(TxR) | ParE_Ser⁴⁴⁹ |
| 5 | E. tarda | Dabcyl-GCTCTCCTTGCAGG-O-K(HEX) | E. tarda |
| | GyrB_Leu⁴⁶⁴ | | GyrB_Leu⁴⁶⁴ |
| 6 | E. tarda | Dabcyl-TGCTATGGGGCGAT-O-K(TxR) | E. tarda |
| | ParC_Gly⁸⁸ | | ParC_Gly⁸⁸ |
| 7 | E. tarda | Dabcyl-TGACAGCGCGGTT-O-K(FAM) | E. tarda |
| | GyrA_Ser⁸³ | | GyrA_Ser⁸³ |
| 8 | E. tarda | Dabcyl-GCGACATCGCCTG-O-K(HEX) | E. tarda |
| | ParC_Ile⁸⁴ | | ParC_Ile⁸⁴ |
| 9 | E. tarda | Dabcyl-CGGTTTATGACACTATCG-O- | E. tarda |
| | GyrA_Asp⁸⁷ | K(TxR) | GyrA_Asp⁸⁷ |

TABLE 6
Information on Nucleotide Sequences of Primers

| | SEQ ID | | | PCR product |
|---|---|---|---|---|
| bacteria species | NOS: | name | Sequence (5′->3′) | size |
| V. harveyi | 10 | gyrA_F | CTTCCAGATGTGCGTGATG | 197 |
| | 11 | gyrA_R | CGAAGTGAGAACGGCTGAG | |
| S. parauberis | 12 | gyrA_F | AGTGTAATCGTGGCCAGAGC | 272 |
| | 13 | gyrA_R | CACCGTCACCATCCATAG | |
| | 14 | parC_F | CGTGATTTGATTACAATGTTG | 203 |
| | 15 | parC_R | CCACTACGGGTTACACTTAC | |
| | 16 | parE_F | CGGCAAGAAGAACAAGAAGG | 340 |
| | 17 | parE_R | ATATGGGCACCATCGGTATC | |
| E. tarda | 18 | gyrA_F | CTGGGCAATGACTGGAAC | 211 |
| | 19 | gyrA_R | GCCATGCGCACTTCGGT | |
| | 20 | gyrB_F | CGTAACCGTAAGAATCAGGC | 170 |
| | 21 | gyrB_R | CTGTGGTAGCGCAGCTTATC | |
| | 22 | parC_F | GTGTATGCGATGTCTGAGCT | 158 |
| | 23 | parC_R | GGATAGCGATAGGAGAAGG | |

Since fluorescence that can be detected simultaneously by a real-time PCR system was limited, three tubes were used for diagnosis, and peptide nucleic acids labeled with different fluorescence dyes were included in different tubes. Specifically, for set 1, SEQ ID NO: 2, SEQ ID NO: 3 and SEQ ID NO: 4 were used; for set 2, SEQ ID NO: 5, SEQ ID NO: 6 and SEQ ID NO: 7 were used; and for set 3, SEQ ID NO: 1, SEQ ID NO: 8 and SEQ ID NO: 9 were used.

Primers were also used for different sets. Specifically, for set 1, SEQ ID NOs: 12 to 17 were used; for set 2, SEQ ID NOs: 18 to 23 were used; and for set 3, SEQ ID NO: 10, SEQ ID NO: 11, SEQ ID NO: 18, SEQ ID NO: 19, SEQ ID NO: 22 and SEQ ID NO: 23 were used.

All the PNA probes used in the present invention were synthesized using a HPLC purification method by Panagene (Korea), and the purities of all the synthesized probes were analyzed by mass spectrometry (the unnecessary secondary structures of the probes were avoided for effective binding to target nucleic acids).

### Example 3: Acquisition of Amplification Curves and Melting Curves Using PNA Probes for Discrimination of Quinolone Antibiotic-Resistant Bacteria

Using the PNA probes and primers of Example 2, amplification curves and melting curves for DNA samples of S. parauberis, E. tarda, and V. harveyi were acquired. These curves were analyzed to discriminate or detect quinolone antibiotic-resistant bacteria.

For experimental use, DNA was extracted from bacteria by use of the QIAamp DNA Mini kit (Qiagen, USA). Amplification was performed using the CFX96™ Real-Time system (BIO-RAD, USA). PCR reactions were simultaneously performed in three tubes. Each PCR reaction mixture had a total volume of 20 μl, and the composition thereof was as follows: 10 μl of 2×qPCR PreMix buffer (Enzynomics, Korea), 0.5 μl of 2 pmol forward primer (each forward primer) and 0.5 μl of 2 pmol reverse primer (each reverse primer), 1 μl of DNA sample, 0.5 μl of 2.5 pmol peptide nucleic acid, 20×SSB buffer (Seasun Biomaterials, Korea), and distilled water.

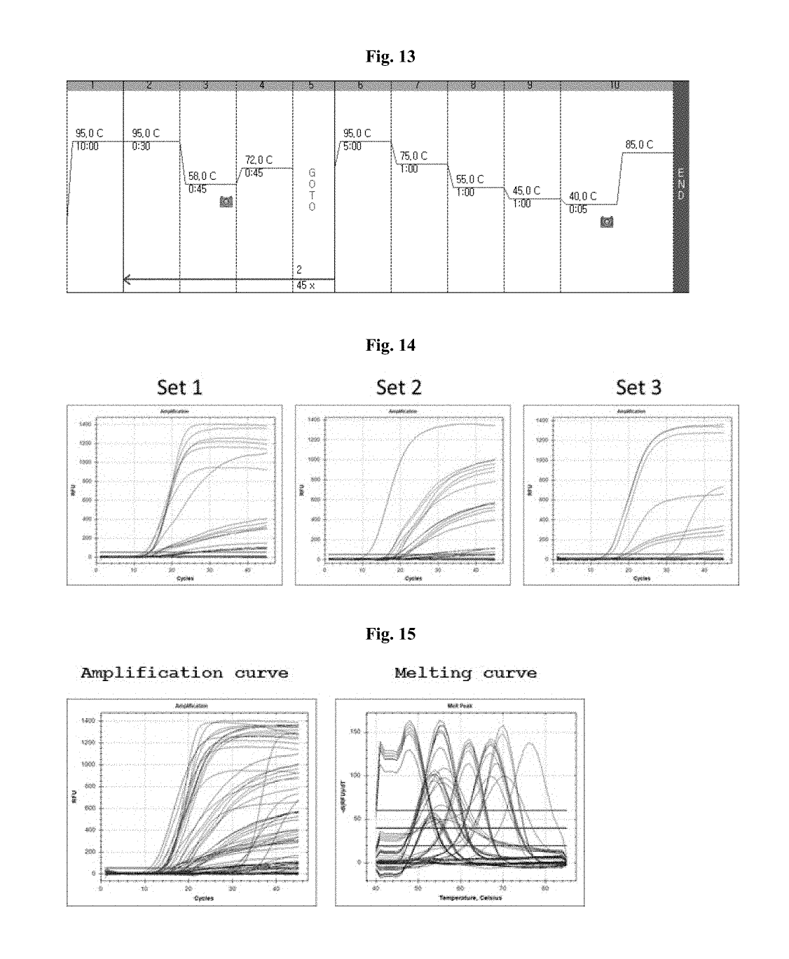

FIG. 13 shows real-time PCR reaction conditions for discrimination and detection of quinolone antibiotic-resistant bacteria. Specifically, FIG. 13 graphically shows a process of amplifying and hybridizing the genetic marker region of quinolone antibiotic resistance gene and increasing the temperature of the hybridized product. Here, the real-time PCR process was performed under the following conditions to obtain an amplification curve: denaturing at 95° C. for 10 min, reacting at 95° C. for 30 sec, 58° C. for 45 sec (fluorescence measurement), and 72° C. for 45 sec and repeating 45 cycles. Next, to hybridize to the PNA probe and obtain a melting curve, melting curve analysis was performed under the following conditions while fluorescence was measured: denaturation at 95° C. for 5 min, and then maintenance at 75° C. for 1 min, 55° C. for 1 min and 45° C. for 1 min, followed by temperature rising from 40° C. to 85° C. at a rate of 1° C. with holding for 5 sec.

The amplification curves are shown in FIG. 14. From the amplification curves, whether or not gene amplification would occur can be determined, and it could be seen that the samples were amplified while showing different fluorescences. The amplification curve and melting curve graphs obtained for sets 1, 2 and 3 according to Example 3 are shown in FIG. 15.

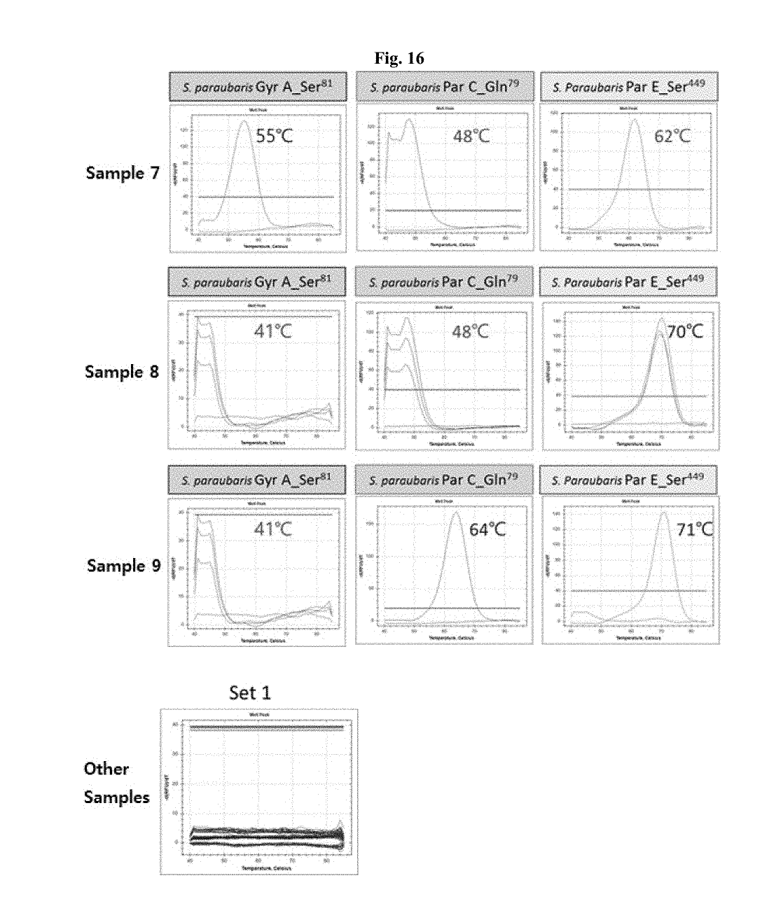

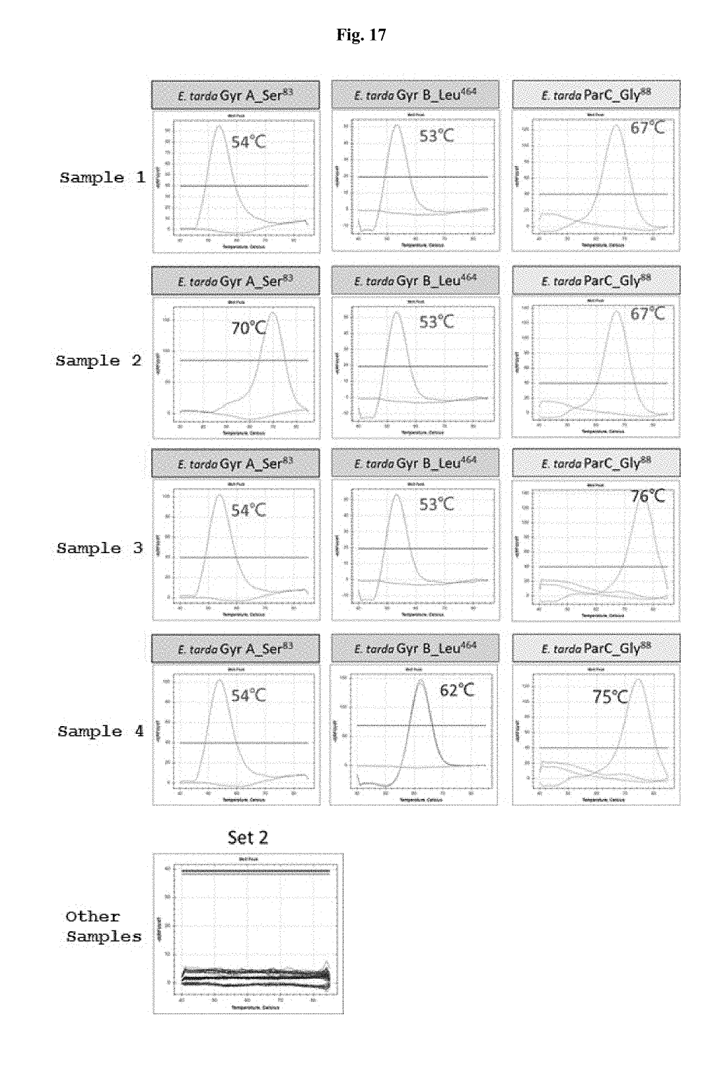

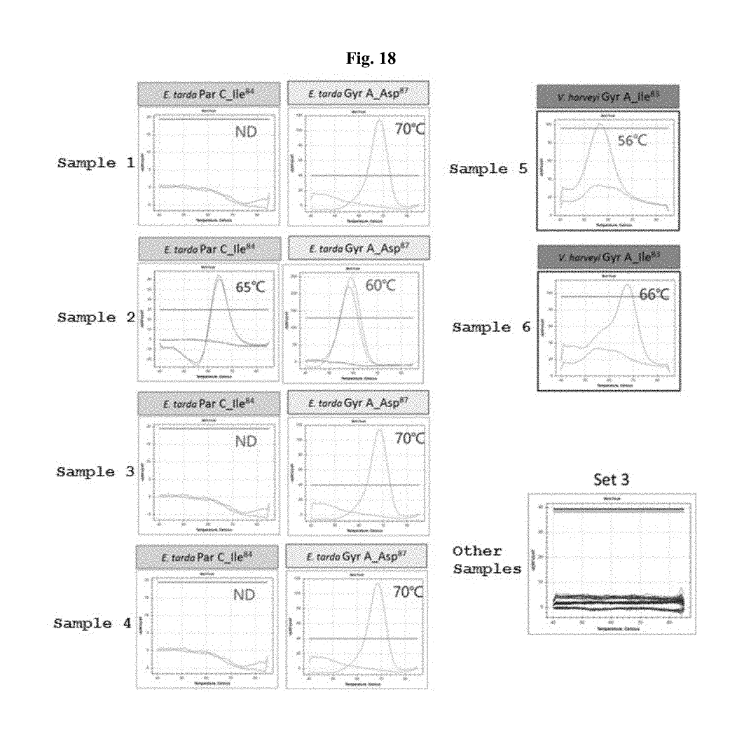

FIG. 16 is a temperature-dependent melting curve graph for each Streptococcus parauberis sample, and shows the results obtained for set 1. Other samples in FIG. 16 refer to Edward tarda and Vibrio harveyi samples. FIG. 17 is a temperature-dependent melting curve graph for each Edward tarda sample, and shows the results obtained for set 2. Other samples in FIG. 17 refer to Streptococcus parauberis and Vibrio harveyi samples. FIG. 18 is a temperature-dependent melting curve graph for each Edward tarda and Vibrio harveyi sample, and shows the results obtained for set 3. Other samples in FIG. 18 refer to Streptococcus parauberis.

### Example 4: Discrimination or Detection of Quinolone Antibiotic-Resistance Genes Based on Melting Fluorescence and Score at Each Temperature

When quinolone antibiotic-resistant genes are to be discriminated or detected using the PNA probes according to the present invention, a table listing scores at different melting temperatures can be previously prepared and can be used.

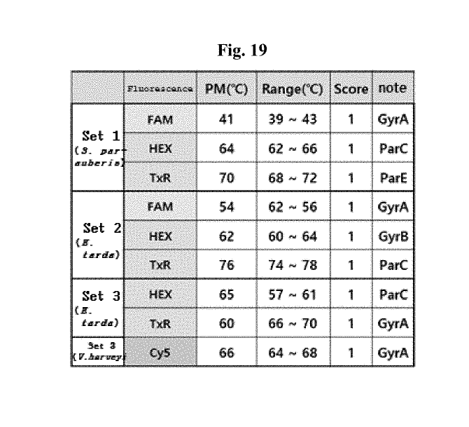

After melting curve analysis was performed as described in Example 3, the obtained fluorescent signal and Tₘ value were digitized according to the temperature at which a perfect match appeared (FIG. 19). Specifically, the range of perfect match temperature ±2° C. is made, and when the Tₘ value for an unknown bacterial DNA sample is within this range, the bacterial sample can be determined to have quinolone antibiotic resistance.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

What is claimed is:

1. A PNA probe for discrimination or detection of quinolone antibiotic-resistant bacteria, which is represented by any one nucleotide sequence selected from the group consisting of SEQ IDS NOs: 1 to 9.

2. The PNA probe of claim 1, wherein the PNA probe comprises any one of a reporter and a quencher attached thereto.

3. The PNA probe of claim 1, wherein the quinolone antibiotic-resistant bacteria is Vibrio harveyi, Streptococcus parauberis, or Edwardsiella tarda.

4. A composition for discrimination or detection of quinolone antibiotic-resistant bacteria, the composition comprising: a primer pair capable of amplifying one or more genes selected from the group consisting of gyrA, gyrB, and parC, which contain a single nucleotide polymorphism (SNP); and the PNA probe of claim 1.

5. The composition of claim 4, wherein the primer pair is a primer pair represented by any one nucleotide sequence pair selected from the group consisting of a nucleotide sequence pair of SEQ ID NOs: 10 and 11, a nucleotide sequence pair of SEQ ID NOs: 12 and 13, a nucleotide sequence pair of SEQ ID NOs: 14 and 15, a nucleotide sequence pair of SEQ ID NOs: 16 and 17, a nucleotide sequence pair of SEQ ID NOs: 18 and 19, a nucleotide sequence pair of SEQ ID NOs: 20 and 21, and a nucleotide sequence pair of SEQ ID NOs: 22 and 23.

6. A kit for discrimination or detection of quinolone antibiotic-resistant bacteria, the kit comprising: a primer pair capable of amplifying one or more genes selected from the group consisting of gyrA, gyrB, and parC, which contain an SNP; and the PNA probe of claim 1.

7. The kit of claim 6, wherein the primer pair is a primer pair represented by any one nucleotide sequence pair selected from the group consisting of a nucleotide sequence pair of SEQ ID NOs: 10 and 11, a nucleotide sequence pair of SEQ ID NOs: 12 and 13, a nucleotide sequence pair of SEQ ID NOs: 14 and 15, a nucleotide sequence pair of SEQ ID NOs: 16 and 17, a nucleotide sequence pair of SEQ ID NOs: 18 and 19, a nucleotide sequence pair of SEQ ID NOs: 20 and 21, and a nucleotide sequence pair of SEQ ID NOs: 22 and 23.

8. A method for discrimination or detection of quinolone antibiotic-resistant bacteria, comprising the steps of:
(a) extracting a target nucleic acid from a sample;
(b) amplifying a quinolone antibiotic-resistant genetic marker nucleotide sequence contained in the target nucleic acid, as a template, by use of a primer pair capable of amplifying one or more genes selected from the group consisting of gyrA, gyrB, and parC, which contain an SNP, and hybridizing the PNA probe of claim 1 to the amplified genetic marker nucleotide sequence;
(c) obtaining a temperature-dependent melting curve while increasing the temperature of the PNA probe-hybridized product resulting from step (b); and
(d) analyzing the melting curve obtained in step (d) to determine a melting temperature, thereby detecting whether or not the bacteria are resistant to the quinolone antibiotic.

9. The method of claim 8, wherein the primer pair is a primer pair represented by any one nucleotide sequence pair selected from the group consisting of a nucleotide sequence pair of SEQ ID NOs: 10 and 11, a nucleotide sequence pair of SEQ ID NOs: 12 and 13, a nucleotide sequence pair of SEQ ID NOs: 14 and 15, a nucleotide sequence pair of SEQ ID NOs: 16 and 17, a nucleotide sequence pair of SEQ ID NOs: 18 and 19, a nucleotide sequence pair of SEQ ID NOs: 20 and 21, and a nucleotide sequence pair of SEQ ID NOs: 22 and 23.
